(19) United States
(12) Patent Application Publication
(10) Pub. No.: US 2013/0345510 A1
(43) Pub. Date: Dec. 26, 2013

(54) METHOD AND ENDOSCOPIC DEVICE FOR EXAMINING OR IMAGING AN INTERIOR SURFACE OF A CORPOREAL CAVITY

(71) Applicant: Ron HADANI, Cresskill, NJ (US)

(72) Inventor: Ron HADANI, Cresskill, NJ (US)

(21) Appl. No.: 13/834,734

(22) Filed: Mar. 15, 2013

Related U.S. Application Data
(63) Continuation-in-part of application No. 13/104,915, filed on May 10, 2011.

(51) Int. Cl. A61B 1/00; A61B 1/06; A61B 1/018

(52) U.S. Cl. CPC A61B 1/00183; A61B 1/018; A61B 1/06
USPC 600/113

(57) ABSTRACT

An auxiliary endoscopic imaging catheter is configured for insertion via a longitudinal working channel of an endoscopic. The endoscopic imaging catheter comprises a longitudinally extending tubular shaft having a proximal end, a distal end, and outer surface, and a longitudinal axis; one or more transparent or translucent elements positioned in the outer surface of the distal end of the shaft; and an imaging system comprising a reflector/refractor element and an imaging sensor, the imaging system being positioned interior of the distal end of the shaft. The reflector/refractor can be rotated or wobbled to obtain images of up to or over 360°. An illumination system is positioned on or in the distal end of the shaft, the illumination system being capable of illuminating a corporeal lumen or organ adjacent to the distal end.

### CROSS-REFERENCE TO RELATED APPLICATION

This application is a continuation-in-part of co-pending, commonly assigned U.S. patent application Ser. No. 13/104,915, filed May 10, 2011, which in turn is based upon and claims the benefit of the filing date of co-pending, commonly assigned U.S. Provisional Patent Application Ser. No. 61/333,214, filed May 10, 2010, incorporated herein by reference, each of which is incorporated herein in its entirety.

### FIELD OF THE INVENTION

The present invention relates to an endoscope assembly. More particularly, the invention relates to an endoscopic imaging catheter with rearward-viewing capability and a method of performing a medical procedure.

### BACKGROUND OF THE INVENTION

An endoscope is a medical or industrial device comprising a flexible or rigid tube and a camera or fiber optics mounted on the distal end or any other part close to the distal end of the tube. The endoscope is insertable into an internal body cavity through a body orifice or a surgical incision to examine the body cavity and tissues as part of a diagnosis or therapeutic procedure such as neurosurgery or head, neck, or spine surgeries. The tube of the endoscope may have no or one or multiple longitudinal channels, which may be used for irrigation or suction, or through which an instrument can reach the body cavity to take samples of suspicious tissues or to perform other surgical procedures such as tissue ablation, sampling, removal, or localized drug delivery.

There are many types of endoscopes, and they are named in relation to the organs or areas with which they are used. For example, rhinoscopes are used for examination and treatment of the nose and brain; gastroscopes are used for examination and treatment of the esophagus, stomach, and duodenum; colonoscopes are used for examination and treatment of the colon; bronchoscopes are used for examination and treatment of the lungs and bronchi; laparoscopes are used for examination and treatment of the peritoneal cavity; sigmoidoscopes are used for examination and treatment of the rectum and the sigmoid colon; arthroscopes are used for examination and treatment of joints; cystoscopes are used for examination and treatment of the urinary bladder; ureteroscopes are used for examination and treatment of the ureters and kidneys; and angioscopes are used for examination and treatment of blood vessels.

Many conventional endoscopes include a single forward-viewing fiber bundle or camera mounted at the distal end of the endoscope that captures and transmits an image to an eyepiece, in the case of fiber bundle, or to a video display monitor at the proximal end, in the case of what is known as videoscope where a miniature camera is mounted at the distal end. The image is used to assist a medical professional in advancing the endoscope into a body cavity and looking for abnormalities. The camera provides the medical professional with a two- and in some scopes three-dimensional (stereoscopic) view from the distal end of the endoscope. To capture an image from a different angle or in a different portion of the endoscope, the endoscope must be maneuvered, repositioned, articulated, or moved back and forth. All these maneuvers of the endoscope prolong the procedure and cause added discomfort, complications, and risks to the patient. Additionally, in certain organs or sites in the body, such as the skull, the brain, the spine, or even the lower gastro-intestinal tract, flexures, tissue folds and unusual geometries of the organ may prevent the endoscope's forward-looking camera from viewing behind tissue folds, flexures, and other “hidden” areas of the organ. The inability to obtain a retrograde view limits a medical professional's ability to view and examine these organs and may even affect the ability to deliver or safely perform a treatment procedure.

### OBJECTS OF THE INVENTION

It is an object of the invention to overcome the problem of limited forward-looking characteristic of known endoscopes.

It is also an object of the invention to provide an auxiliary endoscopic imaging catheter comprising:

a longitudinally extending tubular shaft having a proximal end, a distal end, and an outer surface;

an illumination system positioned on the distal end of the shaft;

one or more transparent or translucent elements positioned in the outer surface of the distal end of the shaft; and

an imaging system comprising a reflector (or refractor) element and a camera sensor positioned interior of the one or more transparent or translucent elements,

wherein the reflector (or refractor) can be rotated to obtain images of up to 360°.

It is a further object of the invention to provide an auxiliary endoscopic imaging catheter comprising:

a longitudinally extending tubular shaft having a proximal end, a distal end, an outer surface, and a longitudinal axis;

an illumination system positioned on the distal end of the shaft;

a transparent or translucent element positioned in the outer surface of the distal end of the shaft; and

an imaging system comprising a reflector (or refractor) and a camera sensor positioned interior of the transparent or translucent element,

wherein the sensor is substantially perpendicular to the longitudinal axis, wherein the reflector (or refractor) is positioned at an angle to the longitudinal axis to reflect images to the image sensor, and wherein the reflector can be rotated to obtain images of up to 360°.

It is a further object of the invention to provide an endoscopic system comprising an endoscope and an endoscopic imaging catheter.

These and other objects of the invention will become more apparent from the discussion below.

### SUMMARY OF THE INVENTION

In one embodiment, the present invention provides for a side view and a rear view that augment the forward view of a conventional endoscope. This invention also provides for a tethered capsule that includes a rearward-looking video camera and a light source, mounted on the distal end of an endoscopic insertion tube that together provide for an auxiliary endoscopic imaging catheter. Further, the endoscopic catheter described herein can be used in applications where the endoscopic catheter is used in a stand-alone fashion as well as in non-medical applications with or without a separate endoscope or device.

Optionally, an auxiliary endoscopic imaging catheter is designed to be insinuated into a channel of a conventional endoscope and to exit from the distal opening of the channel of the conventional endoscope.

According to an embodiment of the invention, the endoscopic device of the invention is an auxiliary endoscopic imaging catheter. The endoscope assembly further includes a main endoscope that includes an insertion tube, a forward-viewing imaging device mounted at a distal end area of the insertion tube, and a channel extending through the insertion tube. The auxiliary endoscopic imaging catheter extends through the channel of the insertion tube and exits from a distal opening of the channel of the insertion tube.

According to another embodiment of the invention, the auxiliary endoscopic imaging catheter is moveable along the channel of the main endoscope and can be rotated or wobbled relative to the channel of the insertion tube.

According to another embodiment of the invention, the auxiliary endoscopic imaging catheter can be moved or advanced along the channel of the main endoscope and the tethered capsule can be rotated or wobbled relative to the endoscopic insertion tube and the channel of the conventional endoscope.

In another embodiment of the invention, the rotation or wobbling of the tethered capsule or the entire auxiliary endoscopic imaging catheter allow for capturing at least a portion of the anterior ethmoid area, where the maxillary and frontal sinuses connect with the nose, or the lumbar vertebra, or a fold, flexure, or other area “hidden” to a forward-looking main endoscope without maneuvering or articulating the tip of the main endoscope.

Optionally, parts or the entire auxiliary endoscopic imaging catheter are disposable or replaceable. Optionally, the tethered capsule is reusable and the auxililiary endoscopic catheter's insertion tube is disposable.

Optionally, the auxiliary endoscopic imaging catheter is flexible and does or does not include a steering or articulation mechanism.

In another embodiment of the invention, an auxiliary endoscopic imaging catheter can be inserted via a longitudinal channel of a main endoscope's insertion tube, the auxiliary endoscopic imaging catheter comprising a tethered capsule which includes a rearward-looking camera and a light source. The tethered capsule is rotatable by a wire connected to a motor connected to a controller, which enables either predefined rotational schemes or manual movements of the capsule by the medical professional or operator.

In another embodiment of the invention, an auxiliary endoscopic imaging catheter for insertion via a longitudinal channel of a main endoscope's insertion tube, comprises a tethered capsule which includes a rearward-looking camera and light source, and the entire auxiliary endoscopic imaging catheter is connected to, and rotated by, a motor connected to a controller, which enables either predefined rotational schemes or manual movements of the capsule by the medical professional or operator.

In another embodiment of the invention, said optical element diverts said optical axis of the camera by approximately 90° in relation to the longitudinal channel of the main endoscope.

Other embodiments of the invention relate to a method and an apparatus for intrabody imaging and, more particularly, but not exclusively, to a method and an endoscope for imaging to the side of the endoscope.

According to other embodiments of the present invention, there is provided an endoscopic imaging catheter which is optionally designed to be inserted via a working channel of an endoscopic insertion tube, and which allows the imaging of walls which encircle an intrabody lumen. Optionally, this catheter is used for imaging portions of the wall which are outside the view of a camera provided with the endoscope insertion tube, for example, portions which are behind the view of such camera.

In another embodiment of the invention, the endoscopic imaging catheter includes a rotatable shaft/wire that is connected to an optical element, such as a mirror, a focusing mirror, a prism, and/or an imaging element, such as an image sensor or fiberoptic image bundle. The rotation, or wobbling, of the shaft allows aiming the optical axis of the imaging element, for example, for imaging a band around the lumen. Optionally, axial motion of the imaging catheter is used to image further bands and/or along a spiral path (e.g., during forward and/or backward motion). Optionally, the insertion tube is not moved during such movement of the insertion catheter.

Optionally, both an insertion tube imager and a catheter imager are used together to image different and possibly overlapping parts of an intrabody lumen, optionally simultaneously. Optionally or alternatively, illumination is shared between the two imaging systems. Optionally, the imaging catheter includes an illumination guide or means. Optionally, such illumination means is arranged so that it does not directly illuminate the imaging system of the imaging catheter. Optionally or alternatively, the imaging catheter imaging system is arranged so that it is not directly illuminated by an illumination means of the endoscope, for example, if the catheter is advanced a sufficient amount. Optionally, this direct illumination is prevented by recessing one or more parts of the imaging system of the imaging catheter so that the body of the imaging catheter blocks light from the illumination means of the endoscope. Optionally or alternatively to change an imaging axis of an imaging element of the imaging catheter, the illumination is reflected, for example, using a mirror, towards the wall of the intrabody lumen, optionally to overlap with a visual field of the endoscopic imaging catheter.

In another embodiment of the invention, the auxiliary endoscopic imaging catheter is covered with a protective sheath, optionally disposable. Such a sheath, which is optionally made from a relatively inexpensive, transparent material such as polyethylene terephthalate (PET) or polycarbonate, allows using the endoscopic imaging catheter in multiple procedures, with multiple patients, without having to perform time consuming disinfective reprocessing procedures. Such a sheath may reduce the price of each one of the procedures. Optionally, such a sheath is designed for a single use to reduce the risk of patient to patient cross contamination. For example, the sheath may tear when removed or may be elastic and provided in everted form or rolled-up for mounting on the imaging catheter.

In another embodiment of the invention, the endoscopic imaging catheter is sized and shaped so that it can be used with a plurality of different endoscope insertion tube designs.

In another embodiment of the invention, the imaging catheter includes an imaging element and an optional image axis changing element (e.g., a mirror, a prism). Optionally, these elements are aligned along the longitudinal axis of the shaft. Optionally, a number of optical elements, such as lenses and diffractive optics elements are positioned between the imaging element and the axis changing element. A potential advantage of such an arrangement is that the diameter of the endoscopic imaging catheter can be made small and/or substantially independent of the length of the optical distance between the imaging element and an imaged area. In other embodiments, the imaging element is pointed at a direction perpendicular to or oblique to the axis of the imaging catheter. An imaging axis changing optical element (e.g., a mirror, a prism) may be included as well.

In other embodiments of the invention, the imaging element is distal to the mirror. In other embodiments the mirror is distal to the imaging elements. Optionally, the catheter is designed so that a line of sight of the imaging system of the imaging catheter exits near a distal end of the catheter. Optionally, this allows more distal imaging without over advancing of the imaging catheter. Optionally or alternatively, positioning of a mirror distally to the imaging element avoids a need to traverse the mirror (or prism) with wires (if any) that connect the imaging element to the outside of the body.

Optionally, the imaging element and/or mirror (or prism) are arranged to simultaneously image on multiple sides of the imaging catheter.

In other embodiments of the invention, the endoscopic imaging catheter is used for scanning the intrabody lumen according to one or more scanning patterns. In another embodiment of the invention, the endoscopic imaging catheter position and/or rotation angle and/or other imaging parameters are optionally automatically controlled by a driver unit that is connected thereto or manually by a physician/operator.

In one embodiment of the invention, an endoscopic imaging catheter for insertion via a longitudinal channel of an endoscopic insertion tube comprises:

an optical element;

an imaging element positioned adjacent the optical element; and

a rotatable shaft at one end of a rotatable wire attached to said optical element, to said imaging element, or to both, and optionally to an illumination source,

wherein rotating or wobbling of said rotatable wire in its other end around its longitudinal axis allows rotating or wobbling of said optical element and/or said imaging element in relation to the other, or rotating or wobbling both said optical element together with imaging element to cover a motion of up to or over 360° around the longitudinal axis of the auxiliary endoscopic imaging catheter, allowing the capture of said side and rear view or at least a portion, of the entire 360° view of a wall encircling an intrabody lumen, which is located behind the imaging element, around the axis of the longitudinal channel.

In one embodiment of the invention, rotation or wobbling allows illumination and capture of at least a portion of an encircling wall of an intrabody lumen or organ.

In one embodiment of the invention, the optical element is proximal in relation to the imaging element when inserted into a longitudinal channel.

In one embodiment of the invention, the optical element is distal in relation to the imaging element when inserted into a longitudinal channel.

In one embodiment of the invention, an endoscopic imaging catheter further comprises an illumination source located proximal to the imaging element, structured and mounted as a 360° ring, thus illuminating the entire side and rear field of view of said imaging element, up to 360° around the longitudinal axis of the endoscopic imaging catheter.

In one embodiment of the invention, an endoscopic imaging catheter further comprises an illumination source located distal to the imaging element, structured and mounted as a 360° ring, thus illuminating the entire side and rear field of view of said imaging element, up to 360° around the longitudinal axis of the endoscopic imaging catheter.

In one embodiment of the invention, an endoscopic imaging catheter further comprises an illumination source which is in-line with the rotating or wobbling imaging element, thus illuminating the approximate field of view captured by the imaging element.

In one embodiment of the invention, an endoscopic imaging catheter further comprises an illumination source which includes a single light source or a plurality of light sources.

In one embodiment of the invention, each light source is an LED.

In one embodiment of the invention, an endoscopic insertion tube is capable of performing a procedure selected from the group consisting of anoscopy, arthroscopy, bronchoscopy, colonoscopy, cystoscopy, esophagogastro-duodenoscopy (EGD), laparoscopy, and sigmoidoscopy.

In one embodiment of the invention, A system comprising the endoscopic imaging catheter of claim 1 and a sheath sized and shaped to cover the endoscopic imaging catheter;

In one embodiment of the invention, A method of probing an intrabody lumen, comprising:

inserting an endoscope insertion tube having a first imaging element mounted on a distal end thereof into an intrabody lumen;

extending a tip of an imaging element from said distal end, said tip comprising a second imaging element and an optical element;

using said first and second imaging elements for respectively capturing first, optionally forward, and second images, optionally side and rear, of an intrabody lumen and a segment of an inner wall encircling said intrabody lumen;

In one embodiment of the invention, The method of claim 12, wherein said first and second images are captured substantially simultaneously.

In one embodiment of the invention, an endoscopic imaging catheter comprises:

a longitudinally extending tubular shaft having a proximal end, a distal end, and an outer surface;

an optical element positioned in the outer surface of the distal end of the shaft; and

an imaging element positioned interior of the optical element,

wherein the proximal end of the shaft can be rotated so that the imaging element in the distal end of the shaft can be rotated up to 360°, and wherein said imaging catheter is capable of being advanced through a biopsy/working channel in an endoscope.

In one embodiment of the invention, rotation allows capturing at least a portion of a wall encircling an intrabody lumen or organ without maneuvering the tip of the endoscopic insertion tube toward said portion.

In one embodiment of the invention, the imaging element precedes the optical element when inserted into a longitudinal channel.

In one embodiment of the invention, the optical element diverts the optical axis by approximately 90° in relation to a longitudinal channel.

In one embodiment of the invention, an endoscopic imaging catheter further comprises an illumination source attached to the rotatable shaft to illuminate a field of view of the imaging device during rotation.

In one embodiment of the invention, an endoscopic imaging catheter further comprises a support construction for fixating said imaging element in relation to the endoscopic insertion tube during said rotation.

In one embodiment of the invention, the optical element is a mirror for adjusting an optical axis of said imaging element.

In one embodiment of the invention, the optical element and the imaging element are aligned in parallel to the rotatable shaft.

In one embodiment of the invention, an endoscopic insertion tube has at least one frontal imaging element attached so as to capture a first image of an intrabody cavity in front of a distal end thereof, the optical element adjusting the optical axis to allow the image sensor to capture a second image of a wall of the intrabody cavity.

In one embodiment of the invention, an endoscope is capable of performing a procedure selected from the group consisting of anoscopy, arthroscopy, bronchoscopy, colonoscopy, cystoscopy, esophagogastro-duodenoscopy (EGD), laparoscopy, and sigmoidoscopy.

In one embodiment of the invention, an endoscopic imaging catheter further comprises a plurality of optical elements and a plurality of imaging elements, the tip being attached to at least one of the plurality of optical elements and the plurality of imaging elements, wherein the rotation changes the rotational angle for each imaging element.

In one embodiment of the invention, an endoscopic imaging catheter configured for insertion via a longitudinal channel of an endoscopic insertion tube, comprises:

an optical element and an imaging element;

a rotatable shaft having a tip attached to at least one of said optical element and said imaging element; and

a sheath sized and shaped to cover said rotatable shaft;

wherein rotation of the rotatable shaft on its axis changes a rotational angle of an optical axis of the imaging element in relation to the longitudinal channel and to the sheath.

In one embodiment of the invention, an endoscopic system comprises:

an endoscope insertion tube having a distal end;

a first imaging element mounted on the distal end to capture a first image of an intrabody lumen;

a rotatable shaft having a tip extended from the distal end and attached to an optical element;

a driver unit for rotating the rotatable shaft according to a predefined scan pattern and

a second imaging element mounted in front of the optical element to capture a second image of an inner wall encircling said intrabody lumen via an optical axis angled by the optical element,

wherein rotation of the rotatable shaft about a shaft axis changes a rotational angle of the optical element in relation to the endoscopic insertion tube to rotate the optical axis about the shaft axis.

In one embodiment of the invention, rotation allows using the second imaging element for imaging more than 50% of the inner wall without maneuvering the insertion tube.

In one embodiment of the invention, rotation allows using the second imaging element for imaging more than 80% of the inner wall without maneuvering the insertion tube.

In one embodiment of the invention, an endoscopic system further comprises a first illumination source positioned to illuminate the intrabody lumen and a second illumination source positioned to illuminate the inner wall.

In one embodiment of the invention, an endoscopic system further comprises an illumination source attached to the tip, the rotation changes a rotational angle of the illumination source to rotate an illumination axis about the shaft axis.

In one embodiment of the invention, an endoscopic system further comprises a driver for performing rotation according to manual instructions received from a user.

In one embodiment of the invention, an endoscopic system further comprises a driver for performing rotation according to a predefined scanning pattern.

In one embodiment of the invention, an endoscopic imaging catheter comprises:

a longitudinally extending tubular shaft having a proximal end, a distal end, and a lumen;

a cylindrical member rotatingly attached to the distal end of the shaft, the cylindrical member having an outer surface with an optical element and an imaging element positioned interior of the optical element;

a motor positioned within the shaft lumen; and

a shaft connecting the motor to the cylindrical member,

wherein the cylindrical member can be rotated so that the imaging element in the cylindrical member can view a field of up to approximately 360° in a direction substantially toward the proximal end of the tubular shaft.

In one embodiment of the invention, a method of probing an intrabody lumen comprises:

inserting an endoscope insertion tube having a first imaging element mounted on a distal end thereof into an intrabody lumen;

extending a tip of an imaging element from said distal end, said tip comprising a second imaging element and an optical element;

using said first and second imaging elements for respectively capturing first and second images of a intrabody lumen and a segment of an inner wall encircling said intrabody lumen; and

rotating at least one of said second imaging element and said optical element about a longitudinal axis of said distal end so as to change a rotational angle of an optical axis of said second imaging element.

In one embodiment of a method of the invention, rotating comprises capturing a plurality of second images of a plurality of consecutive rotational segments of said inner wall.

In one embodiment of a method of the invention, the method further comprises imaging at least 50% of the inner wall according to the plurality of second images without further maneuvering the endoscope insertion tube.

In one embodiment of a method of the invention, the first and second images are captured substantially simultaneously.

In one embodiment of the invention, a method of configuring an endoscopic medical device comprises:

connecting a first imaging element to a distal end of an endoscope insertion tube having a longitudinal channel; and

inserting a shaft having a tip with a second imaging element and an optical element via said longitudinal channel until said tip comes out of said longitudinal channel, at least one of said second imaging element and said optical element being connected to a rotatable shaft traversing said imaging element,

wherein said shaft is rotatable on its axis so as to rotate at least one of said second imaging element and said optical element in relation to the other.

In one embodiment of a method of the invention, the method further comprises covering said shaft with a sheath before said inserting.

In one embodiment of the invention, an endoscopic system comprises:

a longitudinally extending member having a distal end;

a first imaging element mounted on said distal end so as to capture a first image of a desired target;

a rotatable shaft having a tip extended from said distal end and attached to an optical element;

a driver unit for rotating said rotatable shaft according to a predefined scan pattern and

a second imaging element mounted in front of said optical element to capture a second image of an inner surface encircling said target via an optical axis angled by said optical element,

wherein rotation of said rotatable shaft about a shaft axis changes a rotational angle of said optical element in relation to said longitudinally extending member to rotate said optical axis about said shaft axis.

In one embodiment of the invention, the target is an intrabody lumen.

Before at least one embodiment of the invention is explained in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### BRIEF DESCRIPTION OF THE DRAWINGS

FIG. 1 is a schematic illustration of a cross-section of an auxiliary endoscopic, stand-alone, or non-auxiliary imaging catheter according to one embodiment of the invention;

FIG. 2 is a schematic illustration of a view of along the line 2-2 in FIG. 1;

FIG. 3 is a schematic illustration of a cross-section of another embodiment of the invention;

FIG. 4 is a perspective schematic illustration of a distal tip of an endoscope with another embodiment of an auxiliary endoscopic imaging catheter of the invention extending therethrough;

FIG. 5 is a partly cross-sectional schematic illustration of the distal tip of the auxiliary endoscopic imaging catheter shown in FIG. 4;

FIG. 6 is a perspective schematic illustration of a distal tip of an endoscope with a further embodiment of an auxiliary endoscopic imaging catheter of the invention extending therethrough;

FIG. 7 is a partly cross-sectional schematic illustration of the distal tip of the auxiliary endoscopic imaging catheter shown in FIG. 6;

FIG. 8 is a partly cross-sectional schematic illustration of the distal tip of yet another auxiliary endoscopic imaging catheter useful according to the invention; and

FIG. 9 is a partly cross-sectional schematic illustration of the distal tip of another auxiliary endoscopic imaging catheter useful according to the invention.

### DETAILED DESCRIPTION OF THE INVENTION

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

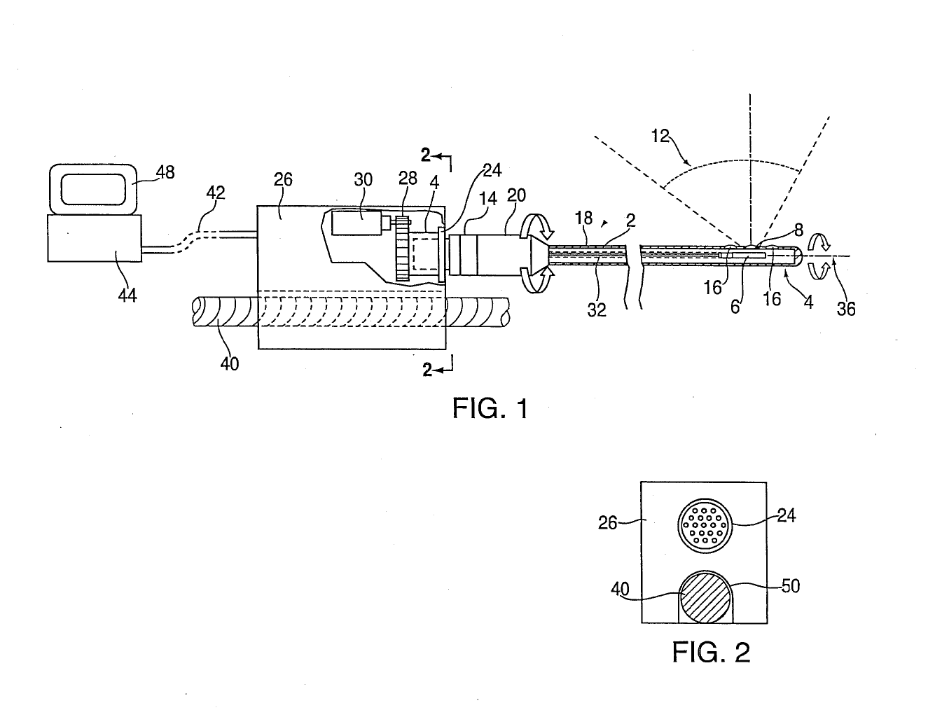

FIG. 1 shows a perspective view of an auxiliary endoscopic imaging catheter system according to one embodiment of the present invention. In this figure the entire auxiliary endoscopic catheter 2 rotates and/or wobbles. Catheter 2 comprises a sealed capsule 4 that comprises a camera 6 and side optical windows 8, which comprise a ring-like shape through the entire circumference of the capsule. Camera 6 is comprised of a single or multiple imaging sensor(s) (as CMOS or CCD) with its electronics as well as certain optical components. Camera 6 is designed to obtain a rearward-looking image field of view 12 which is tilted towards the proximal end 14 of auxiliary endoscopic imaging catheter 2. Capsule 4 also contains a light source, which is comprised of single or multiple LEDs with their side illumination windows 16, which comprise a ring-like shape through the entire circumference of the capsule, or optionally, other types of illumination elements. Capsule 4 is an integral part of an insertion tube or shaft 18. A male (or female) electrical connector 20 at proximal end 14 of auxiliary endoscopic imaging catheter 2 is plugged into a female (or male) electrical connector 24 which is part of a driver unit 26. Female electrical connector 24 is rotatable/wobbled by being linked directly or through gears 28 to a motor 30. Male electrical connector 20 at proximal end 14 of auxiliary endoscopic imaging catheter 2 connects the electronic cabling 32 from camera 6 into driver unit 26. The wobbling motion as opposed to continuing rotation of female electrical connector 24 and male electrical connector 20 prevents the wrapping of electronic cabling within driver unit 26. By connecting auxiliary endoscopic imaging catheter 2 to driver unit 26 (plugging male connector 20 to female electrical connector 24) the motor 30 rotates/wobbles the entire auxiliary endoscopic imaging catheter 2 around its longitudinal axis 36. It rotates/wobbles capsule 4 so a rearward-looking field of view 12 can be obtained practically from 360° around longitudinal axis 36 of auxiliary endoscopic imaging catheter 2.

Driver unit 26 is clamped onto a cable 40 of a main endoscope. A driver cable 42 is connected between driver unit 26 and a camera control unit 44, with a video display 48 that shows the images captured by auxiliary endoscopic imaging catheter 2.

In FIG. 2, a cross-sectional view across line 2-2 in FIG. 1 shows that driver unit 26 has a recess 50 that positions driver unit 26 on cable 40.

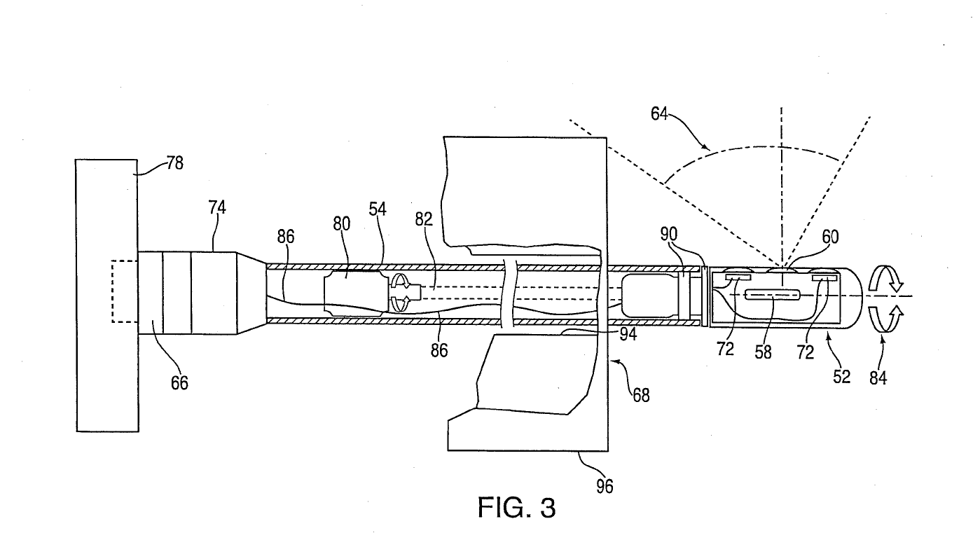

FIG. 3 represents a perspective view of another auxiliary endoscopic imaging catheter system according to an embodiment of the present invention. In this embodiment only sealed capsule 52 rotates/wobbles, whereas insertion tube or shaft 54 remains stationary. Capsule 52 contains a camera 58 with its side optical window 60, which comprises a ring-like shape through the entire circumference of the capsule. Camera 58 is comprised of a single or multiple imaging sensor(s) (such as CMOS or CCD) with its electronics as well as certain optical components. Camera 58 is designed to obtain a rearward-looking image field of view 64 which is tilted towards the proximal end 66 of auxiliary endoscopic imaging catheter 68. Capsule 52 also contains a light source, which is comprised of single or multiple LED(s) with their side illumination window(s) 72, which comprise a ring-like shape through the entire circumference of the capsule, or optionally other illumination elements.

Capsule 52 is secured to insertion shaft 54, for example, with a fine thread, which makes it easy to assemble and disassemble. A male electronic connector 74 at the proximal end 66 of auxiliary endoscopic imaging catheter 68 is plugged into a driver unit 78. While insertion shaft 54 of auxiliary endoscopic imaging catheter 68 is stationary, a capsule 52 wobbles, for instance, +/−360° back and forth, due to the motion from motor 80 imparted to shaft 82 along longitudinal axis 84 that engages capsule 52. The wobbling motion as opposed to continued rotation prevents possible disengagement of capsule 52 and wrapping of the electronic cabling 86. Optionally cable 86 could be routed through shaft 82. Radial shaft seal(s) 90 mounted between capsule 52 and stationary insertion shaft 54, prevent fluids from penetrating into auxiliary endoscopic imaging catheter 68. Electrical connector 74 at proximal end 66 of auxiliary endoscopic imaging catheter 68 carrying the electronic cabling 86, is connected to driver unit 78. Auxiliary endoscopic imaging catheter 68 is advanced distally through a biopsy/working channel 94 in a standard endoscope 96.

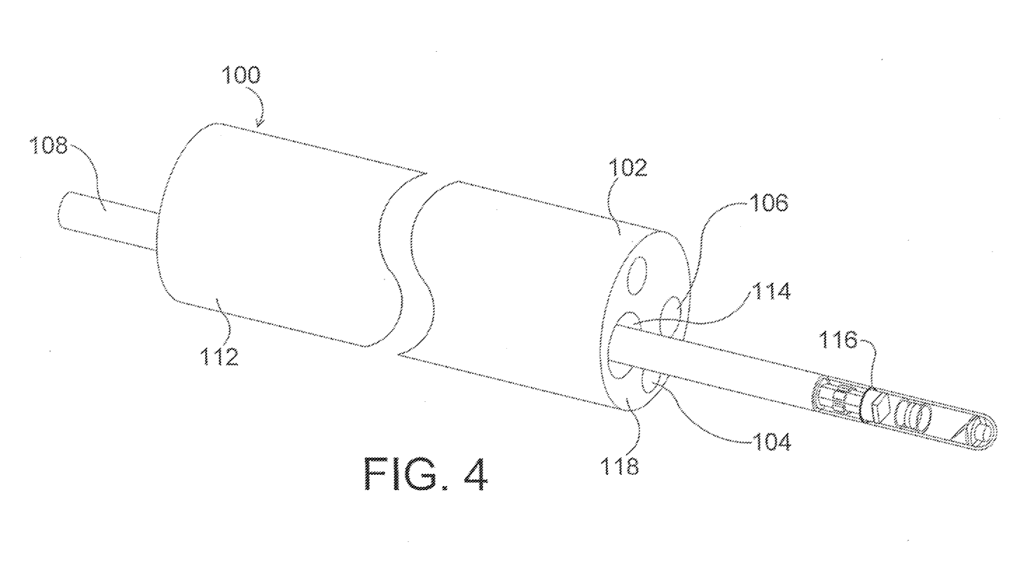

FIG. 4 is a perspective view of a conventional endoscope 100 where the distal tip 102 of endoscope 100 comprises at least one forward-viewing camera 104 and at least one forward-looking illumination source 106. An auxiliary endoscopic imaging catheter 108 extends into the proximal portion 112 of endoscope 100 and through a working channel 114 such that the distal tip or portion 116 of imaging catheter 108 is distal to the distal surface 118 of endoscope 100.

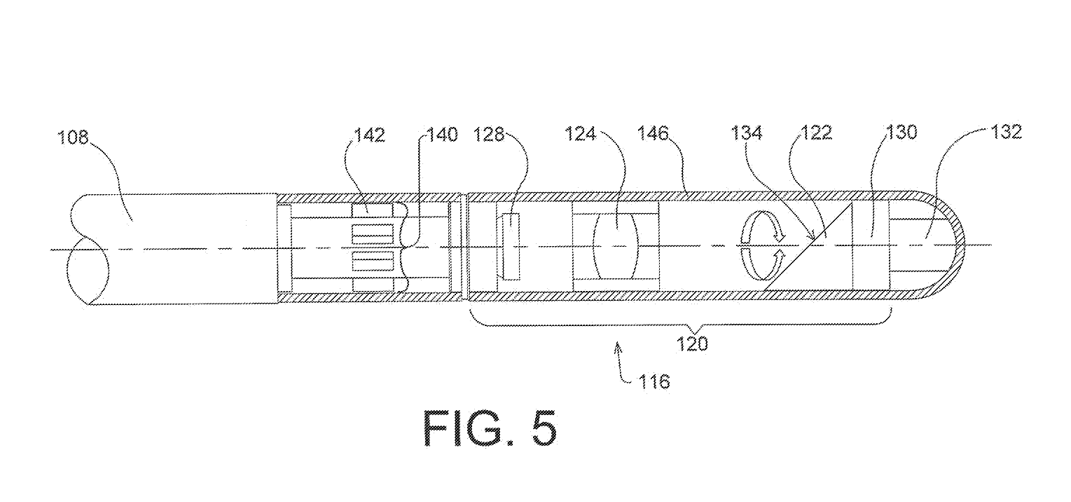

Catheter distal tip 116 is shown in detail in FIG. 5, where a camera system 120 comprises a rotating and/or wobbling reflector (or refractor) 122, stationery optics 124, and a stationery camera or sensor 128. Reflector 122 is attached to a circular mount 130. Mount 130 is engaged with or attached to a motor 132 that rotates mount 130 and reflector 122 together.

Preferably motor 132 is connected to wires (not shown) that extend proximally to a power source (not shown) and a controller (not shown). Optionally motor 132 could be powered by a battery (not shown) or a passive and/or inductive source. Also, motor 132 could be in wireless communication with a controller (not shown). Preferably camera 128 is connected to wires (not shown) that extend proximally to a controller (not shown).

Reflector 122 is a prism that has a flat surface 134 at an angle about 45° to the longitudinal axis of catheter tip 116 and that reflects or refracts light images from areas substantially lateral and/or proximal to catheter distal tip 116 through optics 124 to camera 128. In FIG. 5 reflector 122, rotating mount 130, and motor 132 are shown distal to optics 124 and camera 128. Optionally reflector 122, mount 130, and motor 132 can be proximal to optics 124 and camera 128.

A rearward illumination system 140 comprises two or more lights 142 such as light emitting diodes (LEDs) positioned in an annular configuration within catheter distal tip 116. Individual lights 142 are positioned to direct light mostly in a direction lateral and somewhat proximal to catheter distal tip 116. Here, illumination system 140 is positioned proximal to camera system 120. However, illumination system 140 could be positioned distal to camera system 120.

Catheter distal tip 116 comprises a tubular casing or outer surface 146 that can be rigid or flexible and substantially translucent or transparent at the portions surrounding illumination system 140 and camera system 120. The annular portion or window of casing 146 directly adjacent illumination system 140 must permit light to be transmitted through casing 146 to illuminate a corporeal cavity or organ (not shown), just as the annular portion or window of casing 146 adjacent at least reflector 122 must permit light to enter catheter distal tip 116 to be reflected or refracted.

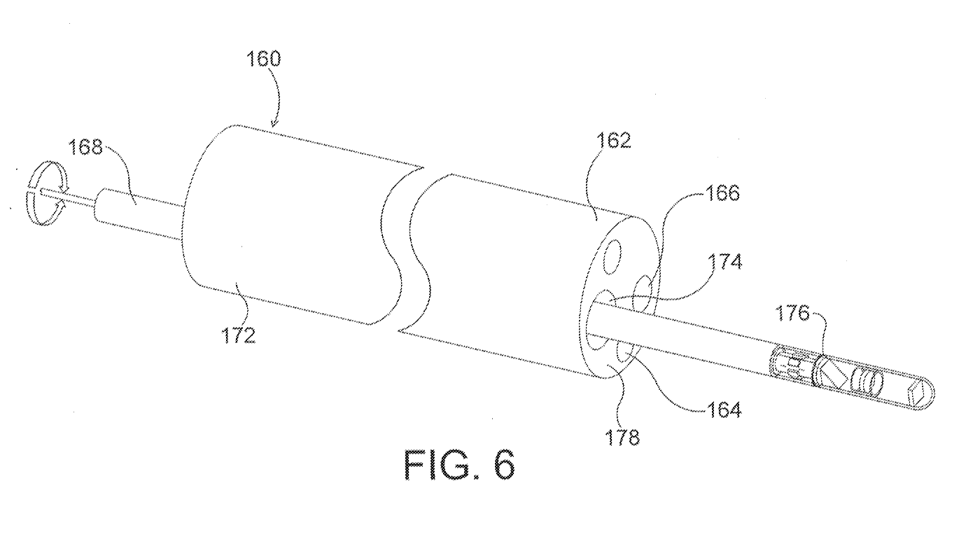

FIG. 6 is a perspective view of a conventional endoscope 160 where the distal tip 162 of endoscope 160 comprises at least one forward-viewing camera 164 and at least one forward-looking illumination source 166. An auxiliary endoscopic imaging catheter 168 extends into the proximal portion 172 of endoscope 100 and through a working channel 174 such that the distal tip or portion 176 of imaging catheter 168 is distal to the distal surface 178 of endoscope 160.

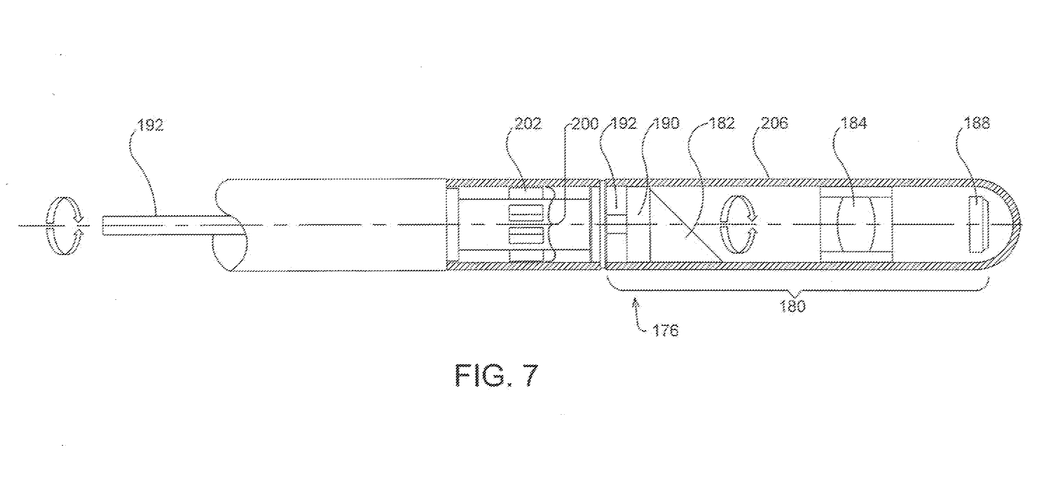

Catheter distal tip 176 is shown in detail in FIG. 7, where a camera system 180 comprises a rotating and/or wobbling reflector 182, stationery optics 184, and a stationery camera or sensor 188. Reflector 182 is attached to a circular mount 190. Mount 190 is attached to a proximally extending rotating and/or wobbling shaft or wire 192 that rotates mount 190 and reflector 182 together.

Preferably camera 188 is connected to wires (not shown) that extend proximally to a controller (not shown). Shaft or wire 192 is connected to a rotating motor (not shown) that may be positioned within or without the distal portion of catheter 168.

Reflector 182 is a prism that reflects or refracts light images from areas substantially lateral and/or proximal to catheter distal tip 176 through optics 184 to camera 188. In FIG. 7 reflector 182, rotating mount 190, and motor 192 are shown distal to optics 184 and camera 188. Optimally reflector 182, mount 190, and motor 192 can be proximal to optics 184 and camera 188.

A rearward illumination system 200 comprises two or more lights 202 such as light emitting diodes (LEDs) positioned in an annular configuration within catheter distal tip 176. Individual lights 202 are positioned to direct light mostly in a direction lateral and somewhat proximal to catheter distal tip 176. Here, illumination system 200 is positioned proximal to camera system 180. However, illumination system 200 could be positioned distal to camera system 180.

Catheter distal tip 176 comprises a tubular casing or outer surface 206 that may be rigid or flexible and substantially translucent or transparent at the portions surrounding illumination system 200 and camera system 180. The annular portion or window of casing 206 directly adjacent illumination system 200 must permit light to illuminate a corporeal cavity or organ (not shown), just as the annular portion or window of casing 206 adjacent at least reflector 182 must permit light to enter catheter distal tip 176 to be reflected or refracted.

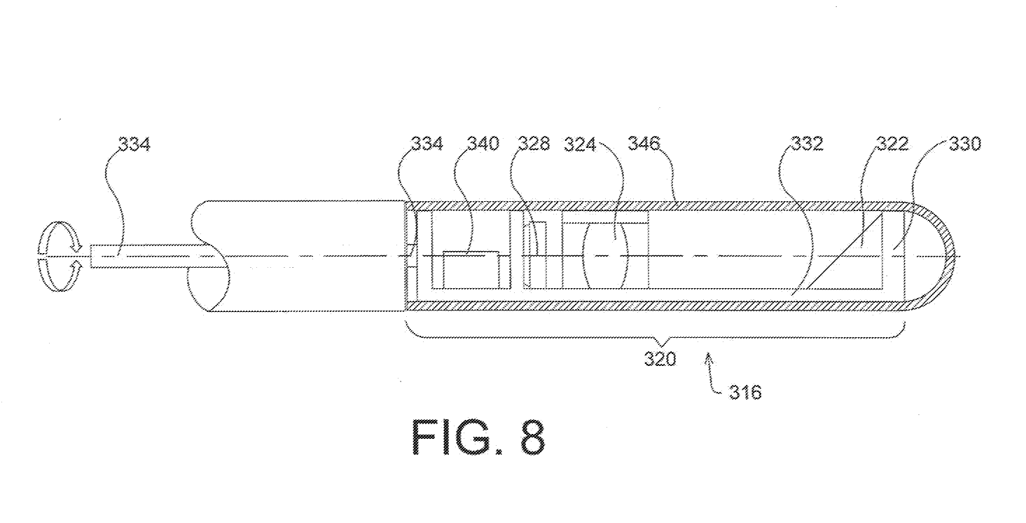

Catheter distal tip 316 is shown in detail in FIG. 8, where a camera system 320 comprises a reflector 322, stationery optics 324, and a stationery camera or sensor 328. Reflector 322 is attached to a circular mount 330. Reflector 322, mount 330, optics 324, and sensor 328 are attached to or embedded in a rotating and/or wobbling shelf or carrier 332. Shelf 332 is attached to rotating and/or wobbling shaft or wire 334. Rotating and/or wobbling carrier 332 may also comprise a rear illumination system 338.

Preferably camera 328 is connected to wires (not shown) that extend proximally to a controller (not shown). Shaft or wire 334 is connected to a rotating motor (not shown) that may be positioned within or without the distal portion (not shown) of the imaging catheter.

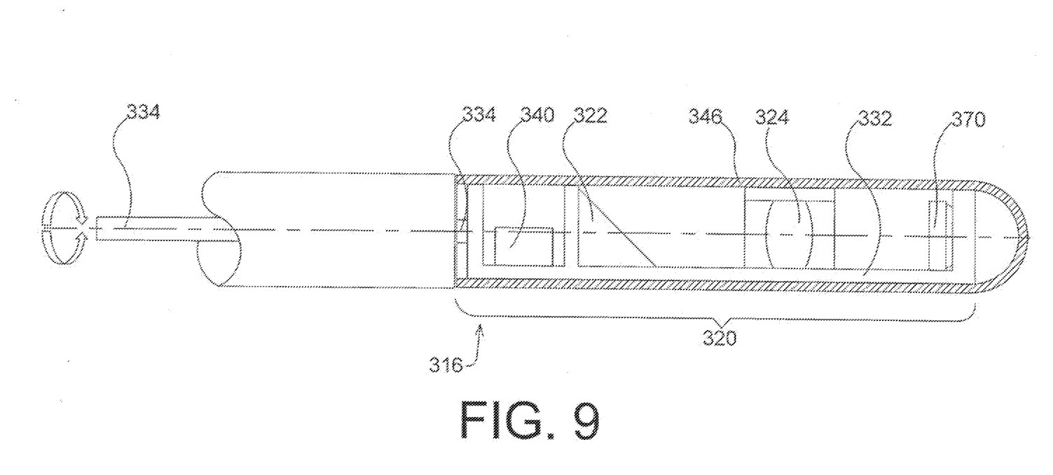

Reflector 322 is a prism that reflects or refracts light images from areas substantially lateral and/or proximal to catheter distal tip 316 through optics 324 to camera 328. In FIG. 8 reflector 322 is shown distal to optics 324 and camera 328. Optionally reflector 322 can be proximal to optics 324 with camera 328 distal to optics 324, as in FIG. 9.

Rearward illumination system 340 may comprise one or more lights such as light emitting diodes (LEDs) positioned within catheter distal tip 316. The light or lights are positioned to direct light mostly in a direction lateral and somewhat proximal to catheter distal tip 316. Here, illumination system 340 is positioned proximal to camera system 320. However, illumination system 340 could be positioned distal to camera system 320.

Catheter distal tip 316 comprises a tubular casing or outer surface 346 that may be flexible or rigid but substantially translucent or transparent at the portions surrounding illumination system 340 and camera system 320. The annular portion or window of casing 346 directly adjacent illumination system 340 must permit light to illuminate a corporeal cavity or organ (not shown), just as the annular portion or window of casing 346 adjacent at least reflector 322 must permit light to enter catheter distal tip 316 to be reflected or refracted.

The description above relates to an endoscopic imaging catheter which is intended for use with a “standard” endoscope, such as endoscope 96, 100, 160, or 220. Such a standard endoscope may be used in various medical procedures in which an intrabody cavity or lumen is imaged, for example, a procedure selected from the group consisting of rhinoscopy, spinal-endoscopy, neuroscopy, sinus-scopy, ENT/Nasal-endoscopy, anoscopy, arthroscopy, bronchoscopy, colonoscopy, cystoscopy, esophagogastro-duodenoscopy (EGD), trans-nasal esophagoscopy (TNE), laryngoscopy, laparoscopy, and sigmoidoscopy.

A standard endoscope includes an insertion tube whose distal section can optionally be articulated, for example, by an articulation knob (or other control), which may be part of an endoscope control/handle unit. Optionally, the control/handle unit is similar to an endoscopic control handle that is incorporated in a conventional endoscope used for intrabody procedures, such as a biopsy. The insertion tube may be detachable from the control/handle unit or in permanent connection. The diameter, length and flexibility of the insertion tube may depend on the procedure for which the endoscope is used. The endoscope may have one or more working channels, for example, for instrumentation, air insufflation, water irrigation, suction and/or light, for example as commonly used in the art. For example, if the insertion tube is used for colonoscopy, the diameter of the working channel which is integral to the insertion tube thereof may be from about 3 mm to about 4 mm, optionally, from about 2.8, 3.2 mm to about 4.2 mm. Optionally, the insertion tube may have varying flexibility over its length.

In one embodiment of the invention, an endoscopic imaging catheter as described above is inserted via a biopsy/working channel, longitudinally traversing the insertion tube through the working channel. In other embodiments, an imaging catheter according to the invention can be integral to the standard endoscope. Optionally, an imaging catheter is sheathed before being inserted into a biopsy/working channel. Optionally or alternatively, a sheath covers the endoscope (or at least an insertion tube) and includes a channel and/or elongate extension for extension of the imaging catheter therethrough.

Optionally, a control handle is provided for manipulation of the imaging catheter, for example, for axial and/or rotational position control and/or for controlling of imaging and/or illumination features thereof. The control/handle unit optionally has a plurality of ports, for example, coupled to the biopsy/working channel, which are in communication with one or more channels in the insertion tube. Each port may allow the insertion of an endoscopic imaging catheter. For example, an endoscopic imaging catheter may be inserted via a biopsy tool port.

Optionally, the insertion tube has an imaging element mounted at a distal end thereof. Exemplary imaging elements include an image sensor, a tip of a fiber optic bundle, a charge coupled device (CCD) based sensor, a complementary metal oxide semiconductor (CMOS) based sensor and/or a radiation sensitive element. For clarity, this imaging element may be referred to herein as a frontal imaging element or as a main imaging element.

Optionally, an insertion tube has an illumination source mounted on the distal end thereof (or provided as a separate movable element, e.g., a catheter), for example, one or morelight emitting diodes (LEDs) or fiberoptic light bundle(s). Optionally, an illumination source illuminates the field of view of the imaging element (and/or of imaging catheter). Optionally, a control unit is used for controlling and/or aiming frontal imaging element and/or illumination source. The cable or another port may be used for providing an illumination channel or fiber bundle that is connected to the frontal illumination source.

Optionally, the auxiliary endoscopic imaging catheter may be extended out past the distal end of the endoscope insertion tube, under visualization of the frontal/main imaging element. This may increase a safety of such extension.

In another embodiment of the invention, rotation is determined according to one or more rotation patterns, each defined to allow automatic scanning of the walls of an intrabody or intracorporeal cavity or lumen. Optionally, the rotation is manually controlled by a physician/operator, for example, by rotation of a handle which is connected to the rotatable catheter shaft 2 or the internal shaft 82. Optionally, the pattern causes rotation of approximately 360° degrees of the optical element 6 or 58 so as to thoroughly scan the walls of the cavity. Another example of a rotation pattern is a wobbling rotation, for example, 170° (or 180° or 200° or 160° or 100° or smaller, larger or intermediate angles) in one direction and then 340° (or 360° or 400° or 200°) back. The wobble in different directions may be asymmetric in magnitude. A driver may include an elastic element that urges the imaging catheter back to a certain rotational position, after being angularly displaced during a wobble (e.g., manually or by a motor). Another example is a rotation during axial movement or with axial movement after each complete rotation, optionally with overlap. The amount of axial motion may be set, for example, according to the lumen diameter.

The optical element may be rotated in relation to the lateral imaging element, for example using a rotatable shaft as described above. This rotation changes the rotational angle of the optical path of a lateral imaging element in relation to the longitudinal axis of a rotatable shaft. Optionally, this allows the lateral imaging element to scan any segment of the walls that encircle the intrabody lumen in the area of the distal end of the insertion tube. Optionally, the optical element may be rotated more than 60°, for example 180°, or 360° around the longitudinal axis of the rotatable shaft. Such a rotation allows capturing more than 50% of the surface area of the inner wall encircling the probed cavity, optionally more than 80%. Optionally, if the rotation is about 360°, imaging of approximately 100% of the surface area of the probed cavity is allowed without maneuvering the tip of the insertion tube of the endoscope 96. As noted above, imaging may proceed other than by complete rotations, for example, the shaft may rotate a plurality or more than one or a fraction of a rotation in one direction and then reverse rotation direction.

The rotation of the optical element through 360° degrees can allow the physician or a medical device to thoroughly probe the surface area of the walls that encircle the intrabody cavities. As used herein, a medical device may comprise a device that performs an automatic analysis or diagnosis based on the images captured by the endoscope. In such a manner, malignancy that is developed on the bottom, the top or the lateral segments of the intrabody lumen's wall may be detected and diagnosed. For example, when an endoscope is used for performing a colonoscopy procedure, the rotating of the optical element to provide a 360° scan allows the physician or the medical device to detect polyps, colorectal neoplasia, ulcerative colitis, colon cancer, and/or other anomalies on any of the tissues that encircle the probed intrabody lumen in the area of the tip of an insertion tube.

Optionally, the rotation of an optical element may be performed automatically, for example using a driver unit that is connected to a rotatable shaft. In such an embodiment, the optical element may be continuously rotated at a fixed and/or a variable pace, to scan walls encircling the probed intrabody lumen. The driver unit may implement different scanning patterns which may be adapted to selected pathologies and/or patients. Optionally, the rotation that is performed by the driver unit and a retraction and/or insertion of the insertion tube into and/or out from the intrabody lumen create a helical scanning pattern. The helical scanning pattern may be adjusted according to pace of the rotation and/or the retraction and/or the insertion.

The simultaneous imaging of the intrabody lumen and the walls that encircle it can provide a complete representation of the probed intrabody area. Further, as the rotation allows scanning the walls of the intrabody lumen without maneuvering the endoscopic imaging catheter, the proficiency level that is needed in order to complete the probing procedure may be reduced. Optionally or alternatively, as the rotation of the optical element may provide a 360° degrees scanning pattern, the procedure may be performed faster.

In some embodiments of the invention, a plurality of lateral imaging elements, and optionally respective optical elements, are used. In such an embodiment, each lateral imaging element, and a respective optical element, is positioned to capture another segment of the encircling wall. In such an embodiment, the cumulative simultaneous field of view of the endoscopic imaging catheter is increased in size. Optionally, the lateral imaging element allows different segments of the optical element to capture opposing (or at a different circumferential displacement angle) segments of the encircling walls. For example, the lateral optical elements can be reflective elements which are respectively positioned in front of a lateral imaging element having two segments. Each one of the lateral optical elements would be positioned in an angle of approximately 45° in relation to the longitudinal axis of a rotatable shaft. In such an embodiment, the rotation of the rotatable shaft by 180° around the longitudinal axis of the rotatable shaft produces a scan pattern that usually covers the entire encircling wall (e.g., ignoring wall folding and the like). Optionally, an optical element is a conical or multi-faceted reflecting element allowing capturing light reflected from 360°. In some embodiments which provide wide angel coverage, such as more than 120°, 180°, 270°, or 360° of a circumference of the intrabody lumen, separate rotation of a catheter may be not provided (e.g., and supported by rotation or articulation of an insertion tube, if needed) or may be attenuated, for example, to less than 360°.

According to some embodiments of the present invention, a reusable and/or a disposable sheath is placed over an endoscopic imaging catheter before insertion thereof into the intrabody lumen. A potential advantage of a disposable sheath is that it allows reusing the endoscopic imaging catheter multiple times. Optionally, a protective sheath is made of a layer of transparent flexible material, such as polyethylene terephthalate (PET), for example, 120-gauge PET, polyvinyl chloride (PVC), Polyethylene terephthalate copolymer (PETG), polyurethane, or other suitable transparent materials. Optionally, a sheath has a transparent segment that covers the distal end of an endoscopic imaging catheter. In such an embodiment, the rotatable shaft allows rotating an optical element without changing the orientation of the sheath. A potential advantage is that, the rotation of an optical element cannot damage the inner walls of an intrabody lumen, e.g., in an embodiment where the optical element is exposed. In some embodiments, with or without a sheath, a window is placed over the optical element. In some embodiments, the entire imaging catheter is rotated for achieving the above described lateral imaging.

Optionally, a lateral imaging element as well as a lateral illumination are connected to an auxiliary CCU and the imaging element of an endoscope is connected to a main CCU. The connection to the auxiliary CCU is optionally performed via a cable that passes through the lumen of an endoscopic imaging catheter. Optionally, the wire is connected thereto via a rotary joint unit that is engaged to allow a communication between the CCU and the lateral imaging element, and optionally the powering thereof, during a rotational motion of the rotatable shaft about its axis. Optionally, the rotary joint unit includes slip rings that maintain electric (and data and/or optical) coupling between the CCU and the lateral imaging element during the rotational motion. Optionally, the slip rings maintain electrical contact between a powering unit and the lateral imaging element during the rotational motion of the rotatable shaft. In another embodiment a magnetically or optically coupled transformer or a wireless (e.g., RF or IR) transmitter replaces the rotary joint unit for transferring imaging signals and power. In some embodiments the cable supports a limited number of rotations of the imaging catheter, after which the catheter is rotated in an opposite direction. Such counter-rotation may be faster if, for example, no image interpretation is being performed on images acquired during such counter rotation.

In another embodiment, the supporting structure supports an imaging element having an optical axis that is directed toward the walls that encircle the cavity. In such an embodiment, flat optics may be used, for example, lenslets and/or diffractive optics elements. Optionally or alternatively, optics are integrated with the imaging element. In some embodiments of the invention, no optical elements other than an imaging element are used.

In some embodiments of the invention, one or more optic fiber or bundle is used for imaging and/or illuminating the walls. In such an embodiment, the fiber optic is inserted via the endoscopic imaging catheter and deflected approximately 90° at the tip thereof, for example, by a suitable inclined surface or channel. The rotation of the optic fiber may allow imaging the walls as described above.

Optionally, an illumination source, such as one or more LEDs, is attached to supporting structure or otherwise optionally rotated to illuminate the field of view of the lateral imaging element. In such an embodiment, each one of the imaging elements has a separate illumination source, which may be controlled and/or activated separately. In such a manner, one illumination source may be dimmed, intensified or turned off, according to an instruction from the physician, providing a better control on the brightness of the captured image. Optionally or alternatively, a plurality of illumination sources are provided so that an entire band of the intrabody lumen is illuminated together.

It is expected that during the life of a patent maturing from this application many relevant systems and methods will be developed and the scope of the term illumination source, optical element, and imaging element is intended to include all such new technologies a priori.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An auxiliary endoscopic imaging catheter for insertion via a longitudinal channel of an endoscopic insertion tube, comprising:
a longitudinally extending tubular shaft having a proximal end, a distal end, and outer surface, and a longitudinal axis;
one or more transparent or translucent elements positioned in the outer surface of the distal end of the shaft; and
an imaging system comprising a reflector/refractor element and a camera sensor, the imaging system being positioned interior of the distal end of the shaft,
wherein the reflector/refractor can be rotated or wobbled to obtain images of up to 360°.

2. The endoscopic imaging catheter of claim 1, wherein an illumination system is positioned on or in the distal end of the shaft, the illumination system being capable of illuminating a corporeal lumen or organ adjacent to the distal end.

3. The endoscopic imaging catheter of claim 2, wherein the illumination system is distal to the imaging system.

4. The endoscopic imaging catheter of claim 2, wherein the illumination system is proximal to the imaging system.

5. The endoscopic imaging catheter of claim 2, wherein an illumination source is structured and mounted as a 360° ring, thus illuminating the entire side and rear field of view of said imaging element, up to or over 360° around the longitudinal axis of the endoscopic imaging catheter.

6. The endoscopic imaging catheter of claim 1, wherein the illumination system comprises an illumination source which includes a single light source or a plurality of light sources.

7. The endoscopic imaging catheter of claim 6, wherein each light source is an LED.

8. The endoscopic imaging catheter of claim 1, wherein the imaging system also comprises optics between the reflector and the camera sensor.

9. The endoscopic imaging catheter of claim 8, wherein the reflector/refractor is distal to the optics and the camera sensor.

10. The endoscopic imaging catheter of claim 8, wherein the reflector/refractor is proximal to the optics and the camera sensor.

11. The endoscopic imaging catheter of claim 8, wherein the camera sensor, the optics, and the reflector/refractor are positioned on and/or attached to a shelf member that can be rotated.

12. The endoscopic imaging catheter of claim 11, wherein the shelf member is attached to or engaged by the distal portion of a longitudinally and proximally extending rotatable shaft member.

13. The endoscopic imaging catheter of claim 12, wherein rotating or wobbling of said rotatable shaft member wire around its longitudinal axis allows the optical element and/or the imaging element to cover a motion of up to or over 360° around the longitudinal axis of the auxiliary endoscopic imaging catheter, allowing the capture of side and rear views or at least a portion, of the entire 360° view of a wall encircling an intrabody lumen.

14. The endoscopic imaging catheter of claim 1, wherein the reflector/refractor is positioned on a circular substrate attached to or engaged by a motor that causes the substrate to rotate.

15. The endoscopic imaging catheter of claim 1, wherein the reflector/refractor is positioned on a substrate attached to or engaged by the distal portion of a longitudinally and proximally extending shaft member that can be rotated.

16. The endoscopic imaging catheter of claim 15, wherein the shaft member has a proximal end that is rotated or wobbled by a motor positioned within or without the tubular shaft.

17. The endoscopic imaging catheter of claim 15, wherein rotating or wobbling of said rotatable shaft member wire around its longitudinal axis allows the optical element and/or the imaging element to cover a motion of up to or over 360° around the longitudinal axis of the auxiliary endoscopic imaging catheter, allowing the capture of side and rear views or at least a portion, of the entire 360° view of a wall encircling an intrabody lumen.

18. The endoscopic imaging catheter of claim 1, wherein the reflector/refractor is a prism having a flat surface at about a 45° angle to the longitudinal axis of the tubular shaft.

19. The endoscopic imaging catheter of claim 1, wherein said rotation or wobbling allows illumination and capture of at least a portion of an encircling wall of an intrabody lumen.

20. The endoscopic imaging catheter of claim 1, wherein said endoscopic insertion tube is capable of performing a procedure selected from the group consisting of anoscopy, arthroscopy, bronchoscopy, colonoscopy, cystoscopy, esophagogastro-duodenoscopy (EGD), laparoscopy, and sigmoidoscopy.

21. A system comprising the endoscopic imaging catheter of claim 1 and a sheath sized and shaped to cover the endoscopic imaging catheter.

22. An endoscope system comprising the endoscopic imaging catheter of claim 1 and an endoscope with a working channel.
